(19) United States
(12) Patent Application Publication
(10) Pub. No.: US 2022/0079614 A1
(43) Pub. Date: Mar. 17, 2022

(54) VASCULAR TREATMENT SYSTEMS AND DEVICES INCLUDING INTRAVASCULAR IMAGING CAPABILITIES

(71) Applicant: KONINKLIJKE PHILIPS N.V., EINDHOVEN (NL)

(72) Inventors: Ryan Michael SOTAK, COLORADO SPRINGS, CO (US); Nathan C. FRANCIS, COLORADO SPRINGS, CO (US); Wade Allen BOWE, COLORADO SPRINGS, CO (US)

(21) Appl. No.: 17/423,318

(22) PCT Filed: Jan. 16, 2020
(86) PCT No.: PCT/EP2020/050958

§ 371 Date: Jul. 15, 2021

Related U.S. Application Data
(60) Provisional application No. 62/794,816, filed on Jan. 21, 2019.

(51) Int. Cl. A61B 17/32; A61B 8/12; A61B 8/00

(52) U.S. Cl. CPC A61B 17/320016; A61B 2017/00243; A61B 8/461; A61B 8/12

(57) ABSTRACT

A vascular treatment system includes a cutting tip, and the cutting tip is configured to be disposed in a treatment space and cut a vascular structure within the treatment space. An imaging device is configured to be disposed in the treatment space and send a signal corresponding to an image of the treatment space. A display is in operative communication with the imaging device and is configured to provide the image of the treatment space to a system user.

### CROSS-REFERENCE TO RELATED APPLICATION

None.

### FIELD OF THE DISCLOSURE

The systems and devices described herein generally relate to vascular treatment systems and devices including intravascular imaging capabilities, and more specifically relate to cardiac lead extraction systems and devices including intravascular imaging capabilities.

### BACKGROUND

Surgically implanted cardiac implantable electronic devices (CIEDs), such as pacemakers and defibrillators, play an important role in the treatment of heart disease. In the 50 years since the first pacemaker was implanted, technology has improved dramatically, and these systems have saved or improved the quality of countless lives. Pacemakers treat slow heart rhythms by increasing the heart rate or by coordinating the heart's contraction for some heart failure patients. Implantable cardioverter-defibrillators stop dangerous rapid heart rhythms by delivering an electric shock.

Some CIEDs typically include a timing device and a lead, which are placed inside the body of a patient. One part of the system is the pulse generator containing electric circuits and a battery, usually placed under the skin on the chest wall beneath the collarbone. To replace the battery, the pulse generator must be changed by a simple surgical procedure every 5 to 10 years. Another part of the system includes the wires, or leads, which run between the pulse generator and the heart. In a pacemaker, these leads allow the device to increase the heart rate by delivering small timed bursts of electric energy to make the heart beat faster. In a defibrillator, the lead has special coils to allow the device to deliver a high-energy shock and convert potentially dangerous rapid rhythms (ventricular tachycardia or fibrillation) back to a normal rhythm. Additionally, the leads may transmit information about the heart's electrical activity to the pacemaker.

For both functions, leads must be in contact with heart tissue. Most leads pass through a vein under the collarbone that connects to the right side of the heart (right atrium and right ventricle). In some cases, a lead is inserted through a vein and guided into a heart chamber where it is attached with the heart. In other instances, a lead is attached to the outside of the heart. To remain attached to the heart muscle, most leads have a fixation mechanism, such as a small screw and/or hooks at the end.

Within a relatively short time after a lead is implanted into the body, the body's natural healing process forms scar tissue along the lead and possibly at its tip, thereby fastening it even more securely in the patient's body. Leads usually last longer than device batteries, so leads are simply reconnected to each new pulse generator (battery) at the time of replacement. Although leads are designed to be implanted permanently in the body, occasionally these leads must be removed, or extracted. Leads may be removed from patients for numerous reasons, including but not limited to, infections, lead age, and lead malfunction.

Removal or extraction of the lead may be difficult. As mentioned above, the body's natural healing process forms scar tissue over and along the lead, and possibly at its tip, thereby encasing at least a portion of the lead and fastening it even more securely in the patient's body. In addition, the lead and/or tissue may become attached to the vasculature wall. Both results may, therefore, increase the difficulty of removing the leads from the patient's vasculature.

A variety of tools have been developed to make lead extraction safer and more successful. Current lead extraction techniques include mechanical traction, mechanical devices, and laser devices. Mechanical traction may be accomplished by inserting a locking stylet into the hollow portion of the lead and then pulling the lead to remove it. An example of such a lead locking device is described and illustrated in U.S. Pat. No. 6,167,315 to Coe et al., which is hereby incorporated herein by reference in its entirety for all that it teaches and for all purposes.

A mechanical device to extract leads may include one or more flexible tubes called sheaths that pass over the lead and/or the surrounding tissue. One of the sheaths may include a tip having a dilator, a separator and/or a cutting blade, such that upon advancement, the tip (and possibly the sheath cooperate to) dilates, separates and/or cuts to separate the scar tissue from other scar tissue including the scar tissue surrounding the lead. In some cases, the tip (and sheath) may also separate the tissue itself from the lead. Once the lead is separated from the surrounding tissue and/or the surrounding tissue is separated from the remaining scar tissue, the lead may be inserted into a hollow lumen of the sheath for removal and/or be removed from the patient's vasculature using some other mechanical devices, such as the mechanical traction device previously described in United States Patent Publication No. 2008/0154293 to Taylor, which is hereby incorporated herein by reference in its entirety for all that it teaches and for all purposes.

Some lead extraction devices include mechanical sheaths that have trigger mechanisms for extending the blade from the distal end of the sheath. An example of such devices and method used to extract leads is described and illustrated in U.S. Pat. No. 5,651,781 to Grace, which is hereby incorporated herein by reference in its entirety for all that it teaches and for all purposes. Another example of these device that has a trigger mechanism for extending the blade from the distal end of the sheath is described and illustrated in United States Patent Publication No. 2014/0277037 having application Ser. No. 13/834,405 filed Mar. 14, 2013, which is hereby incorporated herein by reference in its entirety for all that it teaches and for all purposes.

Lead extraction procedures typically include the use of fluoroscopy to facilitate visualization and tracking of lead extraction devices within a patient's body. However, fluoroscopy has several disadvantages. For example, fluoroscopy provides poor contrast for soft tissues. As another example, fluoroscopy provides two-dimensional imaging of three-dimensional anatomy. These disadvantages inhibit physicians from understanding the anatomy of a specific patient's body. In other cases, lead extraction procedures include the use of an imaging catheter in addition to lead extraction devices. However, such imaging catheters typically require another venous access point and a second operator, and the second operator must attempt to spatially register the lead extraction device to the imaging catheter. Furthermore, imaging catheters are typically poorly suited for lead extraction procedures in terms of, for example, form factor, visual field, and/or accessibility.

Accordingly, it is desirable to provide improved vascular treatment systems and devices including intravascular imaging capabilities.

### SUMMARY

The present disclosure presents a vascular treatment system that includes a cutting tip, and the cutting tip is configured to be disposed in a treatment space and cut a vascular structure within the treatment space. An imaging device is configured to be disposed in the treatment space and send a signal corresponding to an image of the treatment space. A display is in operative communication with the imaging device and is configured to provide the image of the treatment space to a system user.

The system according to the previous paragraph, further comprising a vascular treatment device carrying the cutting tip and the imaging device, and the vascular treatment device detachably coupling to the display.

The system according to any of the previous paragraphs, further comprising a sheath assembly comprising a distal end portion, the cutting tip being rotatably carried at the distal end portion.

The present disclosure also presents a vascular treatment device that includes a cutting tip, and the cutting tip is configured to be disposed in a treatment space and cut a vascular structure within the treatment space. An imaging device is configured to be disposed in the treatment space and send a signal corresponding to an image of the treatment space to a display for providing the image of the treatment space to a device user.

The device according to the previous paragraph, further comprising a sheath assembly comprising a distal end portion, the cutting tip being rotatably carried at the distal end portion.

The device according to any of the previous paragraphs, wherein the imaging device is carried at the distal end portion.

The device according to any of the previous paragraphs, wherein the imaging device is translatably carried by the sheath assembly.

The device according to any of the previous paragraphs, wherein the sheath assembly comprises a longitudinal axis, and the image of the treatment space comprises a viewing centerline substantially parallel to the longitudinal axis.

The device according to any of the previous paragraphs, wherein the sheath assembly comprises a longitudinal axis, and the image of the treatment space comprises a viewing centerline substantially perpendicular to the longitudinal axis.

The device according to any of the previous paragraphs, wherein the imaging device is a first imaging device, further comprising a second imaging device configured to be disposed in the treatment space, wherein the sheath assembly comprises a longitudinal axis, the first imaging device provides the image of the treatment space with a first viewing centerline substantially perpendicular to the longitudinal axis, and the second imaging device provides the image of the treatment space with a second viewing centerline substantially parallel to the longitudinal axis.

The device according to any of the previous paragraphs, wherein the sheath assembly comprises a longitudinal axis, and the image of the treatment space comprises an acute viewing centerline relative to the longitudinal axis.

The device according to any of the previous paragraphs, wherein the imaging device is a first imaging device, further comprising a second imaging device configured to be disposed in the treatment space, the first imaging device and the second imaging device providing the image of the treatment space in a viewing plane.

The device according to any of the previous paragraphs, wherein the viewing plane is a first viewing plane, further comprising a third imaging device and a fourth imaging device configured to be disposed in the treatment space, the third imaging device and the fourth imaging device providing the image of the treatment space in a second viewing plane.

The device according to any of the previous paragraphs, wherein the first viewing plane is substantially perpendicular to the second viewing plane.

The device according to any of the previous paragraphs, wherein the imaging device is an ultrasound device, and further comprising an acoustic lens coupled to the ultrasound device.

The present disclosure also presents a vascular treatment device that includes a sheath assembly. The sheath assembly includes a distal tip and an inner lumen extending proximally within the sheath assembly from the distal tip, wherein the inner lumen is configured to receive a lead. A cutting tip is disposed radially inward of the distal tip, wherein the cutting tip is configured to cut vascular material coupled to the lead. An imaging device is disposed within the distal tip and radially and concentrically outward of the cutting tip, wherein the imaging device sends a signal corresponding to an image of a treatment space adjacent the distal tip.

The device according to the previous paragraph, wherein the cutting tip is rotatable relative to the distal tip.

The device according to any of the previous paragraphs, wherein the cutting tip is translatable relative to the distal tip.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (for example, X₁ and X₂) as well as a combination of elements selected from two or more classes (for example, Y₁ and Zₒ).

The term “a” or “an” entity refers to one or more of that entity. As such, the terms “a” (or “an”), “one or more” and “at least one” may be used interchangeably herein. It is also to be noted that the terms “comprising”, “including”, and “having” may be used interchangeably.

The term “means” as used herein shall be given its broadest possible interpretation in accordance with 35 U.S.C. Section 112(f). Accordingly, a claim incorporating the term “means” shall cover all structures, materials, or acts set forth herein, and all the equivalents thereof. Further, the structures, materials or acts and the equivalents thereof shall include all those described in the summary, brief description of the drawings, detailed description, abstract, and claims themselves.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure may be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a schematic illustration of a vascular treatment system according to an embodiment of the present disclosure.

FIG. 2 is a side view of an exemplary vascular treatment device of vascular treatment systems according to embodiments of the present disclosure.

FIG. 3A is a partial side view of a distal end portion of an exemplary vascular treatment device according to embodiments of the present disclosure.

FIG. 3B is an end view of the distal end portion of the vascular treatment device of FIG. 3A.

FIG. 4A is a partial side view of a distal end portion of another exemplary vascular treatment device according to embodiments of the present disclosure.

FIG. 4B is an end view of the distal end portion of the vascular treatment device of FIG. 4A.

FIG. 5A is a partial side view of a distal end portion of another exemplary vascular treatment device according to embodiments of the present disclosure.

FIG. 5B is an end view of the distal end portion of the vascular treatment device of FIG. 5A.

FIG. 6A is a partial side view of a distal end portion of another exemplary vascular treatment device according to embodiments of the present disclosure.

FIG. 6B is an end view of the distal end portion of the vascular treatment device of FIG. 6A.

FIG. 7A is a partial side view of a distal end portion of another exemplary vascular treatment device according to embodiments of the present disclosure.

FIG. 7B is an end view of the distal end portion of the vascular treatment device of FIG. 7A.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

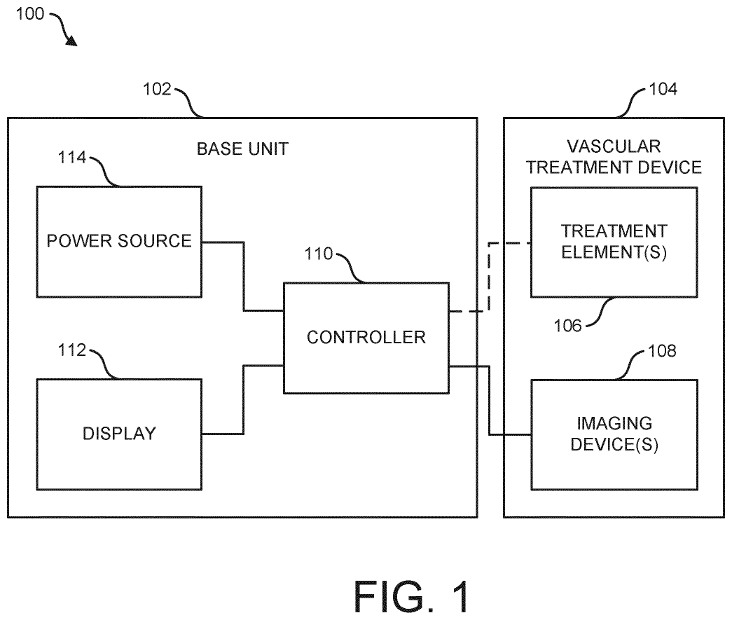

The present disclosure relates generally to vascular treatment systems and devices including intravascular imaging capabilities. FIG. 1 illustrates a vascular treatment system 100 according to an embodiment of the present disclosure. The vascular treatment system 100 generally includes a base unit 102 this is configured to be disposed externally from a treatment space (for example, the vasculature of subject, such as a patient) and a vascular treatment device 104 that is configured to be at least partially disposed within the treatment space and provide treatment to the subject during a vascular surgical procedure. The vascular treatment device 104 may detachably couple to the base unit 102. Similarly, the vascular treatment device 104 may be a “single use” device, and the base unit 102 may be a “multiple use” unit. The vascular treatment device 104 includes one or more treatment elements 106 that interact with and modify vascular structures (for example, tissue, plaque deposits, and the like). The treatment elements 106 may be, for example, configured to physically engage and thereby modify vascular structures (more specifically, the treatment elements 106 may be cutting elements, shearing elements, dilating elements, or the like). As another example, the treatment elements 106 may be configured to emit energy that modifies vascular structures (more specifically, the treatment elements 106 may emit electrical energy or radiofrequency energy, or the treatment elements 106 may be optical fibers that emit laser energy).

The vascular treatment device 104 further includes one or more imaging devices 108 that facilitate providing images of the treatment space to a system user (for example, a physician). The imaging devices 108 may be, for example, ultrasound imaging devices (as more specific examples, piezo-ceramic devices, piezo-film devices, piezoelectric micromachined ultrasonic transducer (PMUT) devices, or capacitive micromachined ultrasonic transducer (CMUT) devices), visible light imaging devices, infrared light imaging devices, spectroscopy imaging devices, impedance mapping imaging devices, or the like. Generally, the imaging devices 108 facilitate providing images of the treatment space to the system user. For example, the imaging devices 108 may send signals from which images of the treatment space may be generated. In some embodiments, the imaging devices 108 may be used in a phased-array manner. In some embodiments, the imaging devices 108 may include a coating to inhibit abrasion of the imaging devices 108 during advancement within a subject. For embodiments in which the imaging devices 108 are optical devices, the coating may be relatively hard and optically clear. For embodiments in which the imaging devices 108 are acoustic devices, the coating may be an acoustic matching layer to the external environment. As specific examples, the coatings may include silicon-based epoxies, polymer-based materials, or the like.

With continued reference to FIG. 1, the base unit 102 includes a controller 110 that is in operative communication with the imaging devices 108 and/or the treatment elements 106 (for example, via wired or wireless communication). The controller 110 is also in operative communication with a display 112 (for example, an LCD display, an LED display, or the like) that provides images of the treatment space. The controller 110 is also in operative communication with a power source 114 (for example, a cord for coupling to the base unit 102 to an external outlet, one or more batteries, or the like), and the controller 110 may thereby deliver power to the imaging devices 108, the treatment elements 106, and/or the display 112. In embodiments in which the treatment elements 106 of vascular treatment devices are optical fibers that emit laser energy, the base unit 102 may also include components for generating laser energy. More specifically, the base unit 102 may be similar to the Spectranetics CVX-300® Excimer Laser System, which is available from the Koninklijke Philips N.V.

Vascular treatment systems according to embodiments of the present disclosure may take other forms. For example, in some embodiments vascular treatment devices may carry one or more of a controller, a display, or a power source. As another example, in some embodiments vascular treatment devices may include combinations of various types of treatment elements and/or imaging devices.

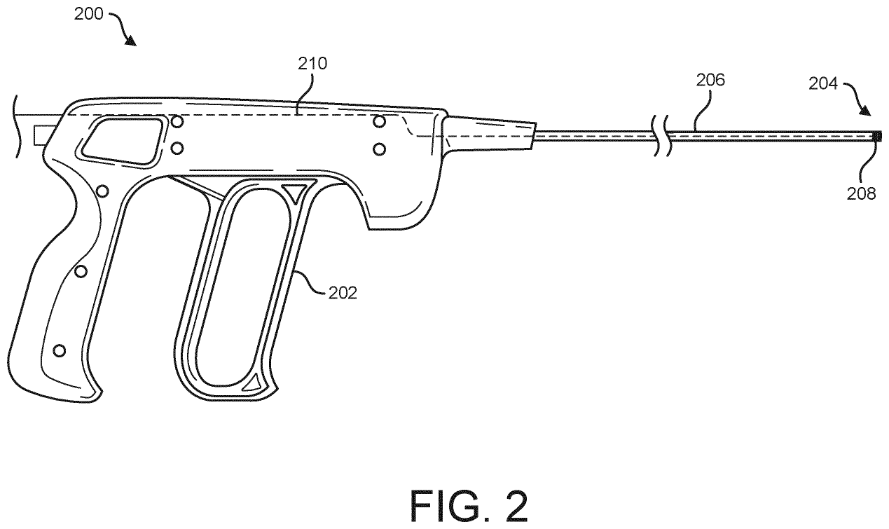

Vascular treatment devices forming part of systems according to embodiments of the present disclosure may take various forms. For example, and referring to FIG. 2, an exemplary embodiment of a vascular treatment device is illustrated. The vascular treatment device is a cardiac lead extraction device 200 and may be similar to any of the extraction devices disclosed in United States Patent Application Publication No. 2017/0172622 having application Ser. No. 15/442,006 filed Feb. 24, 2017 or United States Patent Application Publication No. 2015/0164530 having application Ser. No. 14/635,742 filed Mar. 2, 2015, which is hereby incorporated herein by reference in its entirety for all that it teaches and for all purposes. That is, the lead extraction device 200 includes a trigger 202 that is actuatable to drive a treatment element, specifically a rotatable cutting tip (not shown) disposed at a distal end portion 204 of a sheath assembly 206, and thereby separate tissue from an adjacent lead. In addition, the lead extraction device 200 includes one or more imaging devices 208 disposed at the distal end portion 204 of the sheath assembly 206. The lead extraction device 200 may further include one or more cables 210 for operatively coupling the device (more specifically, the imaging devices 208) to a base unit. Alternatively, the imaging devices 208 may be wirelessly operatively coupled to a base unit. In other embodiments, vascular treatment devices may facilitate removal or manipulation of other indwelling objects (for example, inferior vena cava filters).

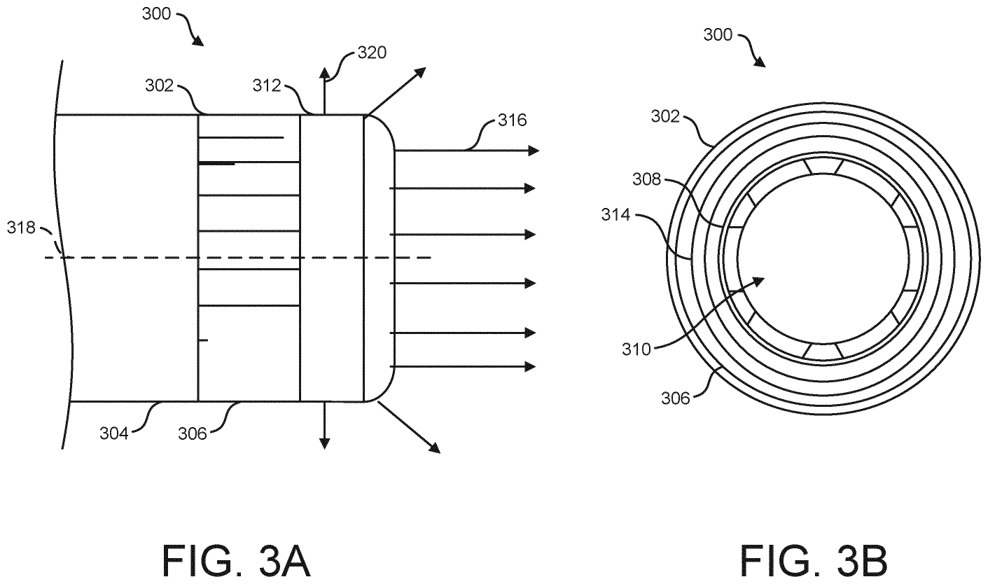

Arrangements of imaging devices and treatment elements of systems and devices according to embodiments of the present disclosure, including the arrangement at the distal end portion of the lead extraction device, may take various forms. For example, and referring to FIGS. 3A and 3B, an exemplary embodiment of a distal end portion 300 of a lead extraction device is illustrated. The distal end portion 300 is part of a sheath assembly 302 that includes an outer sheath 304 or jacket and an outer band or distal tip 306 coupled to and extending distally from the outer sheath 304. An inner sheath (not shown) is rotatably carried within the outer sheath 304, and a cutting tip 308 couples to and extends distally from the inner sheath. As such, the cutting tip 308 is rotatable relative to the outer band 306 to cut and separate tissue from an adjacent lead. The cutting tip 308 may also selectively extend distally relative to the outer band 306 to cut and separate tissue from the lead. The cutting tip 308 and the inner sheath also define an inner lumen 310 for receiving such a lead.

The distal end portion 300 of the lead extraction device further includes a first imaging device 312 (see FIG. 3A) and a second imaging device 314 (see FIG. 3B), which may specifically be any of the imaging devices described herein. Generally, the first imaging device 312 and the second imaging device 314 send signals corresponding to an image of the treatment space, and a display in operative communication with the imaging devices (shown elsewhere) provides the image of the treatment space to a user. The first imaging device 312 is carried by the outer band 306. The first imaging device 312 may have a generally annular shape. The first imaging device 312 may be disposed within the outer band 306 and radially and concentrically outward of the cutting tip 308. The first imaging device 312 may be disposed to provide the image of the treatment space with a first viewing centerline 316 that is substantially perpendicular to a longitudinal axis 318 of the sheath assembly 302 (that is, perpendicular ±5 degrees). Stated another way, the first imaging device 312 may be a transversely-viewing imaging device. The first imaging device 312 may provide a viewing cone of ±45 degrees from the centerline 316. The second imaging device 314 is carried by the outer band 306 distally relative to the first imaging device 312. The second imaging device 314 may have a generally annular shape. The second imaging device 314 may be disposed within the outer band 306 and radially and concentrically outward of the cutting tip 308. The second imaging device 314 may be disposed to provide the image of the treatment space with a second viewing centerline 320 that is substantially parallel to the longitudinal axis 318 (that is, parallel ±5 degrees). Stated another way, the second imaging device 314 may be a distally-viewing imaging device. The second imaging device 314 may provide a viewing cone of ±45 degrees from the centerline 320. In some embodiments, the first imaging device 312 and the second imaging device 314 may be recessed into the outer band 306 to inhibit abrasion of the imaging devices during advancement of the vascular treatment device within a subject. In some embodiments, the distal end portion 300 includes only one of the first imaging device 312 and the second imaging device 314. That is, in some embodiments distal end portions of vascular treatment devices according to the present disclosure include only a distally-viewing imaging device or only a transversely-viewing imaging device.

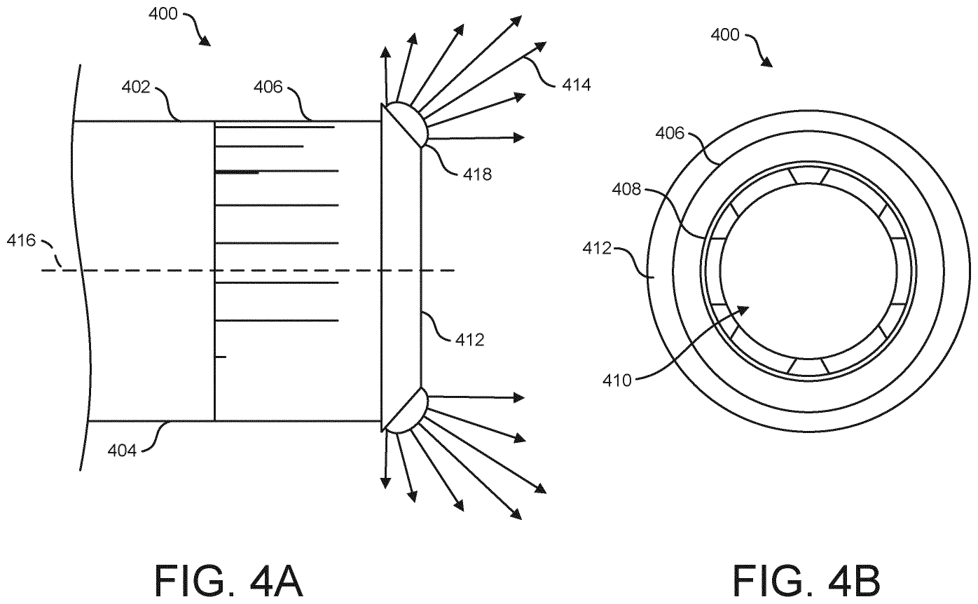

As another example and referring to FIGS. 4A and 4B, an exemplary embodiment of a distal end portion 400 of a lead extraction device is illustrated. The distal end portion 400 is part of a sheath assembly 402 that includes an outer sheath 404 or jacket and an outer band or distal tip 406 coupled to and extending distally from the outer sheath 404. An inner sheath (not shown) is rotatably carried within the outer sheath 404, and a cutting tip 408 couples to and extends distally from the inner sheath. As such, the cutting tip 408 is rotatable relative to the outer band 406 to cut and separate tissue from an adjacent lead. The cutting tip 408 may also selectively extend distally relative to the outer band 406 to cut and separate tissue from the lead. The cutting tip 408 and the inner sheath also define an inner lumen 410 for receiving such a lead.

The distal end portion 400 of the lead extraction device further includes an imaging device 412, which may specifically be any of the imaging devices described herein. Generally, the imaging device 412 send a signal corresponding to an image of the treatment space, and a display in operative communication with the imaging device 412 (shown elsewhere) provides the image of the treatment space to a user. The imaging device 412 is carried on an outer corner of the outer band 406. In some embodiments, the imaging device 412 is flush with the distal end of the outer band 406. More specifically, the imaging device 412 may be mounted to a chamfer (not shown) formed on the outer band 406. In some embodiments, the imaging device 412 is recessed relative to the outer band 406. The imaging device 412 may have a generally annular shape. The imaging device 412 may be disposed to provide the image of the treatment space with an acute viewing centerline 414 relative to a longitudinal axis 416 of the sheath assembly 402. The imaging device 412 may provide a viewing cone of ±45 degrees from the centerline 414. In some embodiments, the imaging device 412 is an ultrasound device, and the distal end portion 400 further includes an acoustic lens 418. Such an acoustic lens 418 facilitates “bending” ultrasound signals that are non-perpendicular to the imaging device 412 into a perpendicular direction relative to the imaging device 412. That is, the acoustic lens 418 facilitates simultaneously providing various viewing angles, such as a viewing angle that is substantially perpendicular to the longitudinal axis 416, a viewing angle along the centerline 414, and a viewing angle that is substantially parallel to the longitudinal axis 416.

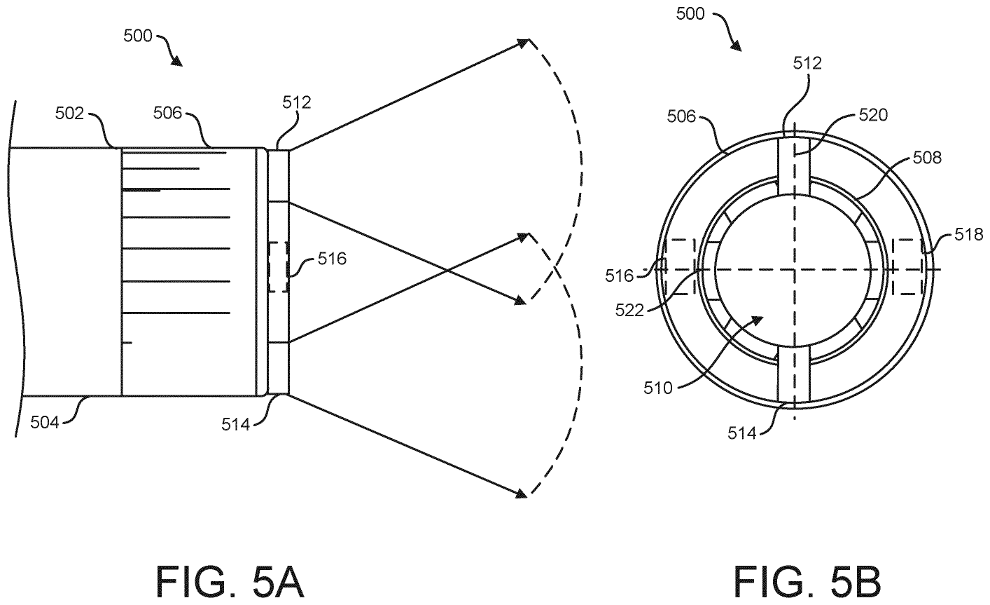

As another example and referring to FIGS. 5A and 5B, an exemplary embodiment of a distal end portion 500 of a lead extraction device is illustrated. The distal end portion 500 is part of a sheath assembly 502 that includes an outer sheath 504 or jacket and an outer band or distal tip 506 coupled to and extending distally from the outer sheath 504. An inner sheath (not shown) is rotatably carried within the outer sheath 504, and a cutting tip 508 couples to and extends distally from the inner sheath. As such, the cutting tip 508 is rotatable relative to the outer band 506 to cut and separate tissue from an adjacent lead. The cutting tip 508 may also selectively extend distally relative to the outer band 506 to cut and separate tissue from the lead. The cutting tip 508 and the inner sheath also define an inner lumen 510 for receiving such a lead.

The distal end portion 500 of the lead extraction device further includes a first imaging device 512, a second imaging device 514, a third imaging device 516, and a fourth imaging device 518, which may specifically be any of the imaging devices described herein. Generally, the imaging devices 512, 514, 516, and 518 send signals corresponding to an image of the treatment space, and a display in operative communication with the imaging devices 512, 514, 516, and 518 (shown elsewhere) provides the image of the treatment space to a user. The imaging devices 512, 514, 516, and 518 are carried by the outer band 506. The first imaging device 512 and the second imaging device 514 are disposed in and provide the image of the treatment space in a first viewing plane 520. The third imaging device 516 and the fourth imaging device 518 are disposed in and provide the image of the treatment space in a second viewing plane 520 that is substantially perpendicular to the first viewing plane 520 (that is, perpendicular ±5 degrees). In some embodiments, the imaging devices 512, 514, 516, and 518 may be recessed into the outer band 506 to inhibit abrasion of the imaging devices 512, 514, 516, and 518 during advancement of the vascular treatment device within a subject. In some embodiments, the distal end portion 500 includes only the first imaging device 512 and the second imaging device 514. The imaging devices 512, 514, 516, and 518 may advantageously require relatively low amounts of power for image acquisition and generation, and the imaging devices 512, 514, 516, and 518 may advantageously require relatively few operative connections to other components, thereby simplifying manufacturing. The imaging devices 512, 514, 516, and 518 may facilitate providing relatively simple images that are easy for a user to understand and interpret.

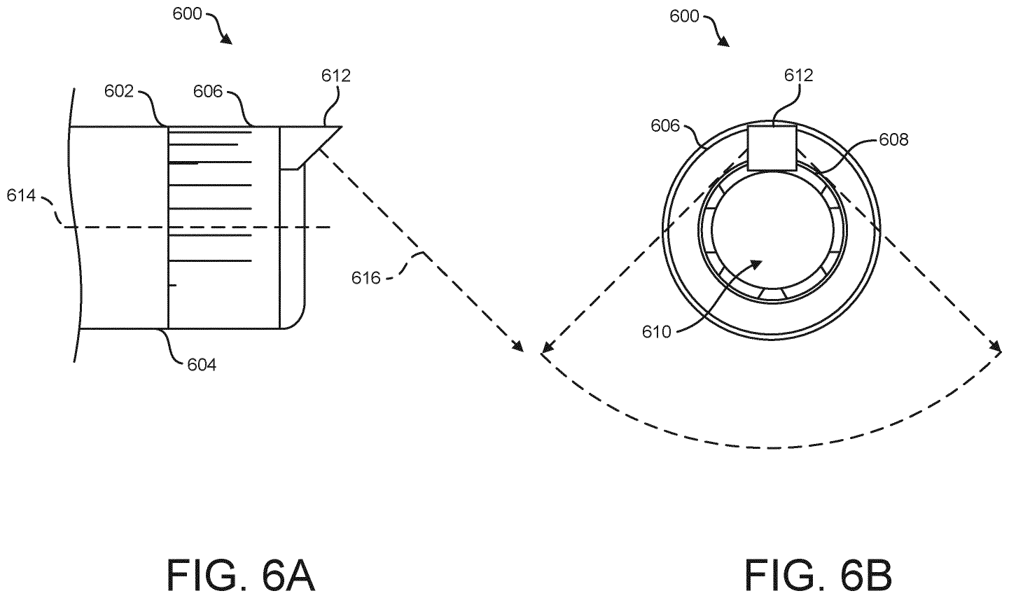

As another example and referring to FIGS. 6A and 6B, an exemplary embodiment of a distal end portion 600 of a lead extraction device is illustrated. The distal end portion 600 is part of a sheath assembly 602 that includes an outer sheath 604 or jacket and an outer band or distal tip 606 coupled to and extending distally from the outer sheath 604. An inner sheath (not shown) is rotatably carried within the outer sheath 604, and a cutting tip 608 couples to and extends distally from the inner sheath. As such, the cutting tip 608 is rotatable relative to the outer band 606 to cut and separate tissue from an adjacent lead. The cutting tip 608 may also selectively extend distally relative to the outer band 606 to cut and separate tissue from the lead. The cutting tip 608 and the inner sheath also define an inner lumen 610 for receiving such a lead.

The distal end portion 600 of the lead extraction device further includes an imaging device 612, which may specifically be any of the imaging devices described herein. Generally, the imaging device 612 send a signal corresponding to an image of the treatment space, and a display in operative communication with the imaging device 612 (shown elsewhere) provides the image of the treatment space to a user. The imaging device 612 has an atraumatic shape that extends distally relative to the outer band 606 and is disposed radially aside of a longitudinal axis 614 of the sheath assembly 602. In some embodiments, the imaging device 612 is partially recessed in the outer band 606. The imaging device 612 may be disposed to provide the image of the treatment space with an acute viewing centerline 616 relative to the longitudinal axis 614 of the sheath assembly 602. The imaging device 612 may provide a viewing cone of ±45 degrees from the centerline 616.

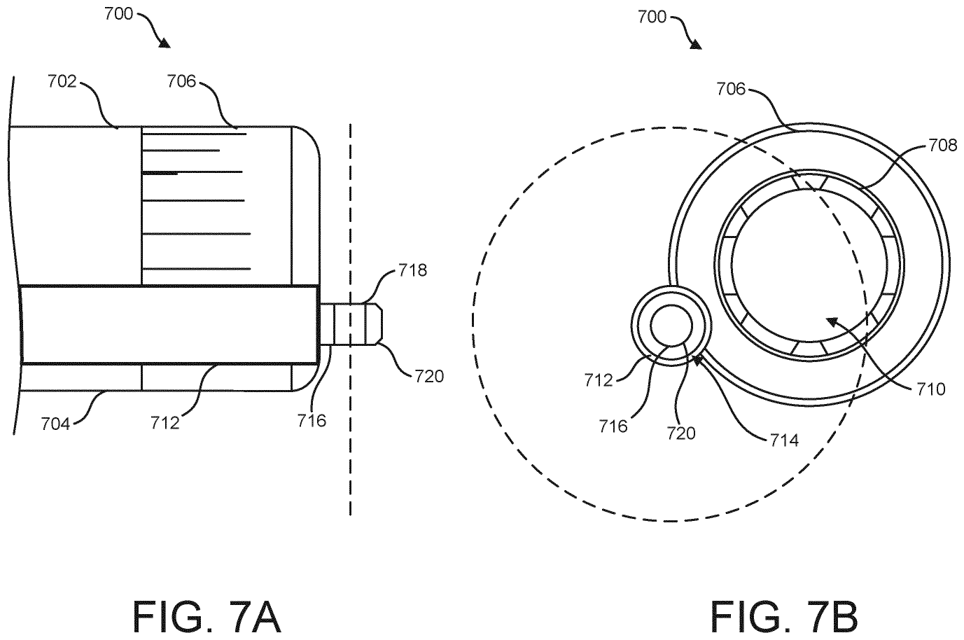

As another example and referring to FIGS. 7A and 7B, an exemplary embodiment of a distal end portion 700 of a lead extraction device is illustrated. The distal end portion 700 is part of a sheath assembly 702 that includes an outer sheath 704 or jacket and an outer band or distal tip 706 coupled to and extending distally from the outer sheath 704. An inner sheath (not shown) is rotatably carried within the outer sheath 704, and a cutting tip 708 couples to and extends distally from the inner sheath. As such, the cutting tip 708 is rotatable relative to the outer band 706 to cut and separate tissue from an adjacent lead. The cutting tip 708 may also selectively extend distally relative to the outer band 706 to cut and separate tissue from the lead. The cutting tip 708 and the inner sheath also define an inner lumen 710 for receiving such a lead.

The sheath assembly 702 of the lead extraction device further includes an auxiliary sheath 712 coupled to the outer sheath 704 and the outer band 706. The auxiliary sheath 712 may be disposed outwardly from the outer sheath 704 and the outer band 706, as illustrated, or inwardly of the outer sheath 704 and the outer band 706. The auxiliary sheath 712 includes an auxiliary lumen 714 that translatably carries an imaging catheter 716. The imaging catheter 716 carries an imaging device 718 at a distal end portion 720. The imaging device 718 may specifically be any of the imaging devices described herein. Generally, the imaging device 718 send a signal corresponding to an image of the treatment space, and a display in operative communication with the imaging device 718 (shown elsewhere) provides the image of the treatment space to a user. The imaging device 718 may be a distally-viewing imaging device, a transversely-viewing imaging device, or both a distally-viewing and transversely-viewing imaging device. In some embodiments, the imaging catheter 716 may include one or more markers and/or fluoroscopy may be used to facilitate registering the imaging device 718 relative to the cutting tip 708. In some embodiments, a mechanical registering mechanism (not shown) may be used to register an imaging plane to the cutting tip 708. In some embodiments, the imaging catheter 716 may be selectively fixable relative to the auxiliary sheath 712.

The foregoing discussion has been presented for purposes of illustration and description. The foregoing is not intended to limit the disclosure to the form or forms disclosed herein. In the foregoing Summary for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, for example, as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A vascular treatment system, comprising:
a cutting tip configured to be disposed in a treatment space and cut a vascular structure within the treatment space;
an imaging device configured to be disposed in the treatment space and send a signal corresponding to an image of the treatment space; and
a display in operative communication with the imaging device and configured to provide the image of the treatment space to a system user.

2. The vascular treatment system of claim 1, further comprising a vascular treatment device carrying the cutting tip and the imaging device, the vascular treatment device detachably coupling to the display.

3. The vascular treatment system of claim 1, further comprising a sheath assembly comprising a distal end portion, the cutting tip being rotatably carried at the distal end portion.

4. A vascular treatment device, comprising:
a cutting tip configured to be disposed in a treatment space and cut a vascular structure within the treatment space; and
an imaging device configured to be disposed in the treatment space and send a signal corresponding to an image of the treatment space to a display for providing the image of the treatment space to a device user.

5. The vascular treatment device of claim 4, further comprising a sheath assembly comprising a distal end portion, the cutting tip being rotatably carried at the distal end portion.

6. The vascular treatment device of claim 5, wherein the imaging device is carried at the distal end portion.

7. The vascular treatment device of claim 5, wherein the imaging device is translatably carried by the sheath assembly.

8. The vascular treatment device of claim 5, wherein the sheath assembly comprises a longitudinal axis, and the image of the treatment space comprises a viewing centerline substantially parallel to the longitudinal axis.

9. The vascular treatment device of claim 5, wherein the sheath assembly comprises a longitudinal axis, and the image of the treatment space comprises a viewing centerline substantially perpendicular to the longitudinal axis.

10. The vascular treatment device of claim 5, wherein the imaging device is a first imaging device, further comprising a second imaging device configured to be disposed in the treatment space, wherein the sheath assembly comprises a longitudinal axis, the first imaging device provides the image of the treatment space with a first viewing centerline substantially perpendicular to the longitudinal axis, and the second imaging device provides the image of the treatment space with a second viewing centerline substantially parallel to the longitudinal axis.

11. The vascular treatment device of claim 5, wherein the sheath assembly comprises a longitudinal axis, and the image of the treatment space comprises an acute viewing centerline relative to the longitudinal axis.

12. The vascular treatment device of claim 4, wherein the imaging device is a first imaging device, further comprising a second imaging device configured to be disposed in the treatment space, the first imaging device and the second imaging device providing the image of the treatment space in a viewing plane.

13. The vascular treatment device of claim 12, wherein the viewing plane is a first viewing plane, further comprising a third imaging device and a fourth imaging device configured to be disposed in the treatment space, the third imaging device and the fourth imaging device providing the image of the treatment space in a second viewing plane.

14. The vascular treatment device of claim 13, wherein the first viewing plane is substantially perpendicular to the second viewing plane.

15. The vascular treatment device of claim 4, wherein the imaging device is an ultrasound device, and further comprising an acoustic lens coupled to the ultrasound device.

16. A vascular treatment device, comprising:
a sheath assembly comprising a distal tip and an inner lumen extending proximally within the sheath assembly from the distal tip, wherein the inner lumen is configured to receive a lead;
a cutting tip disposed radially inward of the distal tip, wherein the cutting tip is configured to cut vascular material coupled to the lead; and
an imaging device disposed within the distal tip and radially and concentrically outward of the cutting tip, wherein the imaging device sends a signal corresponding to an image of a treatment space adjacent the distal tip.

17. The vascular treatment device of claim 16, wherein the cutting tip is rotatable relative to the distal tip.

18. The vascular treatment device of claim 18, wherein the cutting tip is translatable relative to the distal tip.
